(19) United States
(12) Patent Application Publication
(10) Pub. No.: US 2015/0082496 A1
(43) Pub. Date: Mar. 19, 2015

(54) RICE GENE, GS3, EXERTING PRIMARY CONTROL OVER GRAIN LENGTH AND GRAIN WEIGHT

(71) Applicant: HUAZHONG AGRICULTURAL UNIVERSITY, Wuhan (CN)

(72) Inventors: Qifa Zhang, Wuhan (CN); Chuchuan Fan, Wuhan (CN); Yongzhong Xing, Wuhan (CN)

(21) Appl. No.: 14/468,261

(22) Filed: Aug. 25, 2014

Related U.S. Application Data
(62) Division of application No. 12/159,964, filed on Dec. 1, 2008, now abandoned.

(30) Foreign Application Priority Data
Jan. 5, 2006 (CN) 200610018107.4

(51) Int. Cl. C12N 15/82

(52) U.S. Cl. CPC C12N 15/8257
USPC 800/312; 435/320.1; 800/298; 800/314; 800/320.3; 800/320.1; 800/320.2; 800/306; 536/23.6; 435/419; 530/372

(57) ABSTRACT

The present invention relates to an isolated major gene GS3 which regulates grain weight and grain length in the rice and the cloning of said gene. The DNA sequence of GS3 gene is as shown in SEQ ID NO. 1 and is 7883 bp in length. GS3 gene comprises 5 exons and encodes 232 amino acids. It is predicted based on bioinformatics analysis that said protein contains conserved domains including a PEBP-like domain, a transmembrane domain, a cysteine-rich domain of TNFR/NGFR and a VWFC domain. cDNA sequence of said gene is as shown in SEQ ID NO. 2. By sequence alignment between three large grain species and 3 small grain species of rice, it is revealed there is only one common single nucleotide mutation in a 7.9-kb region between the two different grain-length groups. Said nucleotide mutation is located at the second exon of the GS3 gene, in which a cysteine codon (TGC) in the small-grain group is mutated to a termination codon (TGA) in the large-grain group. This mutation causes a premature termination in the large-grain group which leads to a 178-amino acids truncation (including part of the PEBP-like domain and all the other three conserved domains). The present invention also provides methods of producing transgenic plants comprising sequences disclosed herein.

This application is a divisional of co-pending U.S. application Ser. No. 12/159,964, filed Dec. 1, 2008; which is a national stage application under 35 §371 of International Application No. PCT/CN2007/000002 filed Jan. 4, 2007, the entire disclosure of which is specifically incorporated herein by reference, which claims the priority of Chinese Patent Application Ser. No. 200610018107.4, filed Jan. 5, 2006.

### TECHNICAL FIELD

The present invention relates to biotechnology of plants. Particularly, the present invention relates to the gene cloning of GS3, a major QTL regulating grain weight and grain length, which is located in the pericentrometric region of chromosome 3 in rice.

### BACKGROUND OF INVENTION

Grain size of rice is an important economic trait because: (1) grain size is a major determinant of grain weight, which is one of the three components of grain yield, and therefore, grain size is an important trait for yield; (2) grain size is also an important trait of rice appearance because grain weight is positively correlated with several characters including grain length, grain width and grain thickness (Evans, 1972, Rice Breeding, Los Banos, International Rice Research Institute, Manila, pp. 499-511). In China, the USA and some Asian countries, Indica rice with long and slender grain is generally preferred by most consumers. In China, a length/width ratio of 2.8 is adopted as an enforced threshold for a national standard for high quality rice. Thus, understanding the genetic basis and molecular mechanisms of grain size is important in improving both rice yield and quality.

In addition, grain size also plays an important role in the study of evolution of cultivars. It is generally believed that wild type relatives are usually small and round in shape, and are thus favored under natural selection. After a long-term domestication and selection by humans, the shape of grain particles has changed significantly. Therefore, the study of the genetic basis of grain size provides clues for the study of the evolution of cultivars.

Grain size is a typical quantitative trait and is complex in its genetic basis. Utilization of molecular marker technology is able to separate and locate QTL (Quantitative Trait Loci) controlling quantitative traits, thus separating the complex quantitative traits to simple Mendelian factors for studies. By using aforesaid methods, many QTLs regulating rice grain size were identified in recent years. Among these QTLs, a major QTL (referred to as GS3 in the present invention) located in the pericentrometric region of rice chromosome 3 was detected by many researchers (Huang et al., 1997, Mol. Breed. 3:105-113; Redoñ a and Mackill, 1998, Theor. Appl. Genet. 96:957-963; Kubo et al., 2001, Rice Genet. Newsl. 18:26-28; Thomson et al., 2003, Theor. Appl. Genet. 107:479-493; Aluko et al., 2004, Theor. Appl. Genet. 109:630-639). Using F_{2:3} and a recombinant inbred line population derived from a cross between Zhenshan 97 and Minghui 63, the present invention has detected a major QTL several times that is present in the GS3 locus regulating both the grain length and the grain weight. This QTL explains over 55% of the total variation of grain length, as well as approximately 20% of the total variation of grain weight (Yu et al., 1997, Proc. Natl. Acad. Sci. USA 94:9226-9231; Li et al., 2000, Theor. Appl. Genet. 101:248-254; Tan et al., 2000, Theor. Appl. Genet. 101:823-829; Xing et al., 2001, Acta. Bot. Sin. 43:721-726; Xing et al., 2002, Theor. Appl. Genet. 105:248-257; Hua et al., 2002, Genetics 162:1885-1895). These results indicate that GS3 gene can be stably expressed in various genetic backgrounds and different environments. Therefore, GS3 gene is greatly potent and a good prospect for the improvement of both yield and quality traits of rice. The high resolution mapping of GS3 gene and the cloning of the corresponding gene provide new genetic resources for the improvement of both yield and quality in rice breeding.

High resolution mapping of the quantitative traits in common population groups is very difficult because it is not easy to determine whether a major QTL or multiple minor QTL are detected (Yano et al., 1997, Plant Molecular Biology 35:145-153) in said population groups. Secondly, in said population groups, multiple QTLs affecting the same trait are separated, therefore the interference caused and the affects of the environmental factors greatly limit the resolution of the location of a QTL. Special population groups can be established from high resolution mapping of a QTL. A common approach is to establish a near isogenic line (NIL) of an interesting QTL and to eliminate most background differences besides the interesting QTL locus to make said QTL presented as typical Mendelian genetics. Said approach has played an important role in many high resolution mapping and gene cloning of QTLs. Li et al. mapped GS3 to a region of 93.8-kb in length (Li et al., 2004, Genetics 168:2187-2195). Said results have provided a basis for the separation of mapping and the cloning of the GS3 gene.

Upon aforesaid purposes, the present inventors have isolated and cloned a major gene GS3 regulating grain weight and grain length in rice by the approach of mapping and cloning, therefore providing new genetic resources for the improvement of both yield and quality in rice breeding, and also providing clues for the study on the evolution of cultivars.

### DETAILED DESCRIPTION OF THE INVENTION

The present invention relates to the isolation and cloning of the whole DNA fragment encoding a major gene regulating both grain weight and grain length in rice. The present invention also relates to the improvements in both yield and quality of rice using said gene. Said gene is named GS3.

The invention established a near isogenic line (NIL) of GS3. GS3 was finely mapped to a region of 7.9-kb in length using the approach of mapping and cloning. With the help of the sequence information of a whole cDNA in aforesaid region, the invention predicted and analyzed the gene structure of GS3 and the protein encoded by GS3. It was found that GS3 comprises 5 exons and encodes 232 amino acids. It was predicted based on bioinformatics that said protein contains conserved domains including a PEBP-like domain, a transmembrane domain, a cysteine-rich domain of TNFR (tumour necrosis factor receptor)/NGFR (nerve growth factor receptor) and a VWFC (von Willebrand factor type C) homologous domain. In addition, the inventors sequenced and compared three large grain species (Minghui 63, a indica rice in China; H94, from Shanghai Agrobiological Gene Center; 93-11, a rice variety that has been completely sequenced) and 3 small grain species (Zhenshan97, a indica rice in China; Chun 7, from Shanghai Agrobiological Gene Center; Niponbare, a rice variety that has been completely sequenced). It was found that there was only a single base variation in said region of 7.9-kb in length between large grain species and small grain species. Said variation was located in the end of the second exon of GS3, wherein a cysteine codon (TGC) in small grain species is mutated to the terminator codon TGA in large grain species. Said mutation causes GS3 to be prematurely terminated in large grain species and leads to the loss of 178 amino acids, including part of the PEBP-like domain and three other conserved domains. It was found through bioinformatics analysis that the VWFC domain regulates growth and development signals in combination with the growth regulator TGF-β family members. In the large grain species, GS3 lacks the VWFC domain, therefore it loses its capacity to regulate growth and development signaling, which finally leads to the change in grain length and grain weight.

The present invention has the following advantages:

(1) it is the first cloning of a gene that highly affects the grain length and grain weight in rice, therefore providing new gene resources for high yield and good quality in rice breeding, and providing a good technology example for the cloning of homologous genes in other species;
(2) the gene cloned in the present invention provides evidence for the study of domestication and molecular evolution of rice and other species.

### DESCRIPTION OF FIGURES

FIG. 1 shows the technical flowchart of the present invention;

FIG. 2 shows the six cultivars used in sequence alignment;

FIG. 3 shows the frequency distribution of 1,000-grain weight, grain length, grain width and grain thickness in the random BC₃ F₂ subpopulation; wherein

(a) frequency distribution of 1,000-grain weight;
(b) frequency distribution of grain length;
(c) frequency distribution of grain width;
(d) frequency distribution of grain thickness;

FIG. 4 shows the map of the GS3 locus on the molecular linkage map of chromosome 3 (unit in cM);

FIG. 5 shows the maps of the gene region, wherein

(a) The high-resolution map of the GS3 gene. The numbers between molecular markers indicate the numbers of recombination events detected between the GS3 locus and respective markers. Genebank no. OSJNBa0030J19 is a BAC clone of Nipponbare encompassing the GS3 locus;
(b) Organization of the GS3 gene. The positions of exons (black boxes), 5′ and 3′ UTR (hatched boxes), translation start codon (ATG), translation stop codon (TGA), and one common single nucleotide mutation in the second exon between the two grain-length groups are indicated, in which a substitution of cysteine (TGC) in the small grain group mutated to the translation stop codon (TGA) in the large grain group;
(c) Predicted sequence of the GS3 gene expression product. The position of the amino acid change in large grain group (cysteine to stop codon) is indicated by an asterisk. The PEBP-like domain is indicated by dashed underline, the transmembrane region by single solid underline, the TNFR/NGFR family cysteine-rich domain by double underline, and the VWFC domain is boxed;

FIG. 6 shows the organization of the predicted GS3 protein indicating the localization of the various conserved domains including the PEBP-like domain, the transmembrane domains, the cysteine-rich domain of TNFR/NGFR and the VWFC homologous domain, wherein the PEBP-like domain is located inside the membrane, while the cysteine-rich domain of TNFR and the VWFC homologous domain are located outside the membrane.

### EXAMPLES

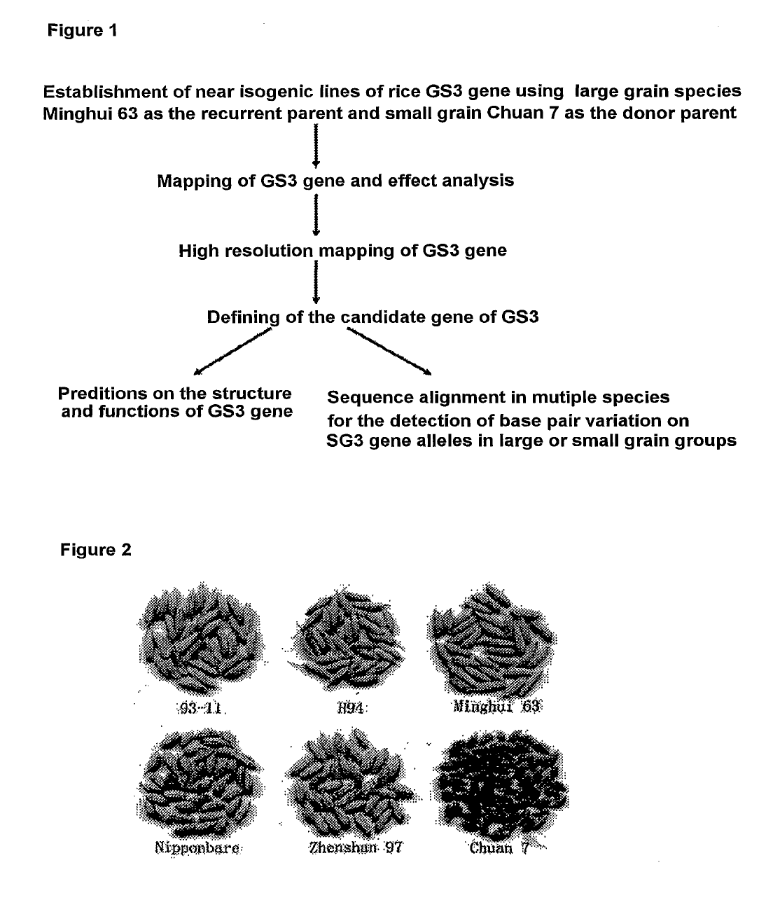

A near isogenic line of rice GS3 gene was established using Minghui 63 as the recurrent parent and Chuan 7 as the donor parent. Mapping and effect evaluation of GS3 gene were carried out on a random BC₃ F₂ subpopulation consisting of 201 BC₃F₂ individuals. By further analysis on 1,384 BC₃F₂ individuals with large grain phenotype using CAPS markers, GS3 was finally mapped between the two CAPS markers GS63 and SF19 (designed by the inventors; see Table 2), which was approximately 7.9 kb in distance. Based on a whole length cDNA sequence, the GS3 gene structure was predicted. In addition, the possible function of GS3 gene was predicted based on the bioinformational technology. It was found by sequence alignment of the 7.9 kb fragment that a mutation was presented in all the large grain species which led to deficiency of 178 amino acids. Such deficiency could lead to the loss of the gene function. It implied the essential reason of the change in grain size, and at the same time, proved the correctness of setting GS3 as the target gene (FIG. 1).

The following Examples further define the present invention and describe the methods for the isolation and cloning of GS3 gene, as well as the methods for the detection of the base mutation between GS3 alleles by sequence alignment. Based on the following recitation and the Examples, the skilled in the art are able to confirm the essential features of the present invention, and are able to make various changes and adjustments on the present invention to apply it on different uses and conditions without deviating from the concepts and scope of the present invention.

### Example 1

### Establishment of a Near Isogenic Lines of Rice GS3 Gene

### 1. Backcrossing and Screening

As shown in FIG. 2, successive crossing was carried out using Minghui 63 (large grain) as the recurrent parent and Chuan 7 (small grain) as the donor parent. Positive selection of GS3 was carried out in F₁, BC₁F₁ and BC₂F₁, that is, selecting individual plants whose targeting region was Minghui63/Chuan7 heterozygous genotype for the following backcrossing. The targeting region was determined in a region between two known SSR (Simple Sequence Repeat) markers RM282 and RM16. In BC₃F₁, in addition to positive selection, surveillance was also carried out in the genetic backgrounds besides the targeting region. Individual plants with a genetic background closest to Minghui63 were selected for the following experiments. Referring to the published rice genetic linkage map (Temnykh et al., 2000, Theor. Appl. Genet. 100:697-712; Temnykh et al., 2001, Genome Res. 11:1441-1452), 125 SSR markers with known polymorphism in the parents and evenly distributed on the 12 rice chromosomes were selected for surveillance of genetic backgrounds. One plant (BC₃F₁-19) was finally selected, whose genotype in the RM282 and RM16 region was Minghui63/Chuan7 heterozygous genotype, while whose genotype in said 125 SSR markers, only about 20% (25 pairs) of the markers were Minghui63/Chuan7 heterozygous genotype, and the rest thereof were all Mingui63 homozygous genotype. The progeny (BC₃F₂ and BC₃F₃) of said individual plant were used in the following experiments.

### 2. SSR Methods

The standard PCR protocol followed the methods taught in Sambrook, J. et al., Molecular Cloning: A Laboratory Manual, 3rd ed., Translated by Jin Dong Yan et al., Science Press. In the PCR, a 20 μl reaction system was used, which contained: 20-50 ng DNA template, 10 mM Tris-HCl, 50 mM KCl, 0.1% Triton X-100, 1.8 mM MgCl₂, 0.1 mM dNTP, 0.2 μM primers (primers of RM282 and RM16 as mentioned above) and 1 U Taq DNA polymerase. Conditions for PCR included: 94° C. predenature for 4 min; 94° C. 1 min, 55° C. 1 min, 72° C. 1 min, 34 cycles; 72° C. elongation 10 min PCR products were separated on a 6% acrylamide gel and then silver-stained (Bassam et al., 1991, Anal. Biochem. 196: 80-83).

### Example 2

### Mapping and Effect Evaluation of GS3 in the Random Subpopulation

### 1. Measurements of Traits of Large and Small Grain

Grain particles were air-dried and stored at room temperature for at least 3 months before testing in order to make sure the dryness and water contents thereof were relatively identical. Ten randomly chosen full filled grains from each plant were lined up closely in a way that each lay head to head, tail to tail, with no overlap and no gap in between. Said grains were arranged length-wise to measure the grain length using a vernier caliper, and then were lined up closely side by side, that is, arranged by breadth to measure the grain width using a vernier caliper. Grain thickness was determined for each grain individually using a vernier caliper, and the values were averaged and used as the measurements for the plant. Grain weight was calculated based on 200 randomly chosen fully filled grains and converted to 1,000-grain weight.

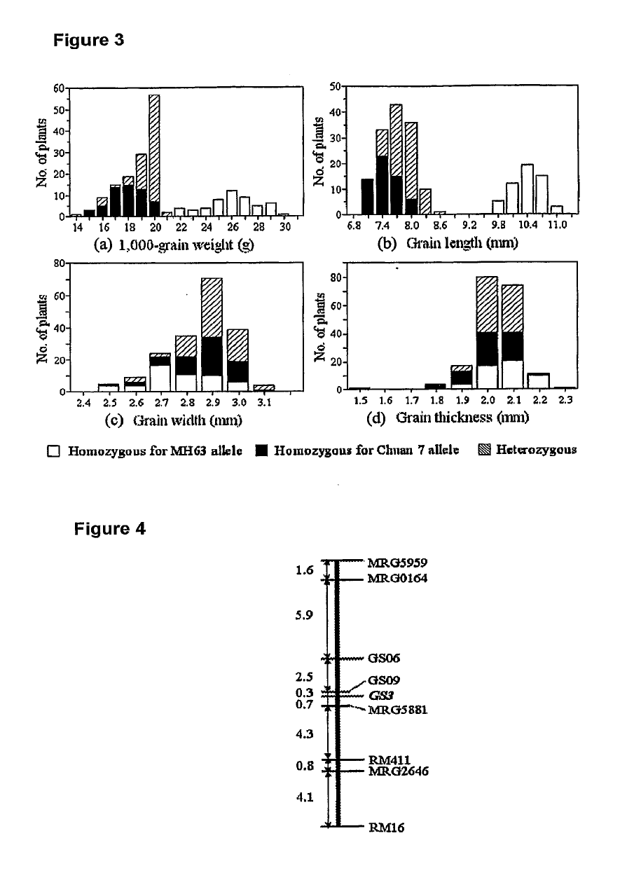

201 individuals of BC₃F₂ derived from the BC₃F₁-19 individual plant were randomly selected to form a random subpopulation. Distributions of grain weight, grain length, grain width and grain thickness were studied in Minhui 63, Chuan 7 and said random subpopulation of BC₃F₂. It was found that all the traits were significantly different between the two parents (Table 1). In the random subpopulation, both grain length and 1,000-grain weight expressed a discontinuous distribution. The plants were classified as long grain and short grain based on a boundary of 8.50-9.50 mm in grain length or 20.5-21.5 g in 1,000-grain weight (Table 1 and FIG. 3). Grain length concurred completely with grain weight, such that long grains were heavier than short grains, and vice visa. However, grain width and thickness showed normal distributions (FIG. 3). For simplicity, in the present invention the large and heavier grains are referred to as long grain, and the opposite type as short grain.

TABLE 1
Descriptive statistics of the traits for the two parents and the long grains and short grains in the random subpopulation

| | Parent (mean ± SD) | | MM | | CC | | MC | |
|---|---|---|---|---|---|---|---|---|
| Trait | Minghui 63 | Chuan 7 | Mean ± SD | Range | Mean ± SD | Range | Mean ± SD | Range |
| 1,000-grain weight (g) | 28.6 ± 0.6 | 12.5 ± 0.4 | 25.6 ± 2.0 | 21.5-29.8 | 17.5 ± 1.3 | 14.2-20.0 | 19.0 ± 1.2 | 14.0-20.5 |
| Grain length (mm) | 9.91 ± 0.099 | 6.30 ± 0.089 | 10.25 ± 0.29 | 9.64-10.73 | 7.32 ± 0.26 | 6.86-7.84 | 7.72 ± 0.25 | 7.24-8.50 |
| Grain width (mm) | 2.80 ± 0.03 | 2.48 ± 0.02 | 2.72 ± 0.13 | 2.43-2.96 | 2.82 ± 0.12 | 2.45-3.06 | 2.85 ± 0.09 | 2.56-3.04 |
| Grain thickness (mm) | 2.13 ± 0.06 | 1.68 ± 0.03 | 2.03 ± 0.09 | 1.81-2.21 | 1.95 ± 0.10 | 1.45-2.10 | 1.99 ± 0.06 | 1.79-2.13 |

### 2. Design of Molecular Markers

Some SSR markers used in the present invention are publicly known. In addition, 11 pairs of Indel (Insert/Deletion) and CAPS (cleaved amplified polymorphic sequence) markers which shows polymorphism between Minghui 63 and Chuan 7 were designed based on the genome DNA sequence of Japonica Rice, Nipponbare and India Rice, 93-11. Said Indel and CAPS markers were used in the high resolution mapping analysis of GS3. The DNA sequences of said markers are listed in Table 2.

TABLE 2
Indel and CAPS markers (primers) developed for the mapping of the GS3 gene locus

| | | | | Annealing | |
|---|---|---|---|---|---|
| | | | | Temp. | Restriction |
| Marker | Type | Forward primer (5′-3′) | Reverse primer (5′-3′) | (° C.) | enzyme |
| GS06 | Indel | AGCAAAGCTGGAACGAAGAG | TAAATTACGCCGTGTCAACG | 55 | |
| | | (SEQ ID NO. 4) | (SEQ ID NO. 5) | | |
| GS09 | Indel | GCAACCAAGTCCACGCTAAT | TAGCCGAAGATCAGCCTCCT | 57 | |
| | | (SEQ ID NO. 6) | (SEQ ID NO. 7) | | |
| GS47 | CAPS | GATTATTGGAGACGGGACGA | GACGGCATGACCACTCTTTT | 55 | HapII |
| | | (SEQ ID NO. 8) | (SEQ ID NO. 9) | | |
| GS52 | CAPS | AGCTTTGGTGTCGTTCTGCT | CCGACTTGGAGAGAATGGAA | 55 | BglI |
| | | (SEQ ID NO. 10) | (SEQ ID NO. 11) | | |
| GS56 | CAPS | GCTGTGTTGTCCTTTGCTGA | CCAATAAACCCCACTGCAAC | 55 | BglI |
| | | (SEQ ID NO. 12) | (SEQ ID NO. 13) | | |
| GS61 | CAPS | CTTTACAAAACCGGCGGTAA | TGAAGCGGACCTAGCATTTT | 53 | BclI |
| | | (SEQ ID NO. 14) | (SEQ ID NO. 15) | | |
| GS63 | CAPS | AAGAACGACTACGCGCATCT | CCATCGCTCTCTTTCCTCAG | 53 | HhaI |
| | | (SEQ ID NO. 16) | (SEQ ID NO. 17) | | |
| GS64 | CAPS | CAACACCAGCAACGAACAAC | ACGAGGGATTATCAGCCATT | 55 | EcoRI, |
| | | (SEQ ID NO. 18) | (SEQ ID NO. 19) | | HapII |
| GS65 | CAPS | CGGTATGCCAAGTTGAATGA | TTGCCGCAGTAAACAAGAAG | 55 | HhaI, |
| | | (SEQ ID NO. 20) | (SEQ ID NO. 21) | | HapII |
| SF18 | CAPS | CCTTCAGTAAGAGAGATGTG | AGTTGATGGTTTTGTGGGAT | 57 | BclI |
| | | (SEQ ID NO. 22) | (SEQ ID NO. 23) | | |
| SF19 | CAPS | TCTGCTTGCGGTTATCTGTA | TTAGGTCCCTTTTCTCGTCC | 57 | SacI |
| | | (SEQ ID NO. 24) | (SEQ ID NO. 25) | | |

Remark:
Markers (primers) GS63 and SF19 were designed by the inventors.

### 3. QTL Mapping and Effect Evaluation of GS3

Said random subpopulation was subjected to genotype analysis using 6 SSR markers (MRG5959, MRG0164, MRG5881, MRG2646, RM411, RM16) and 2 Indel markers (G506 and G509). Mapmaker/Exp 3.0 (Lincoln et al. 1992, Whitehead Institute Technical Report, Whitehead Institute, Cambridge, Mass., USA) was used to establish a partial genetic linkage map in GS3 region. QTL analysis on the traits including grain length, width, thickness and 1,000-grain weight of the random subpopulation was conducted using the program Mapmaker/QTL 1.1 at a threshold of LOD 3.0. QTL analysis indicated that a QTL found in the interval between GS09 and MRG5881 had effects simultaneously on grain weight, grain length, grain width and grain thickness, and contributed 83.4%, 95.6%, 19.8% and 12.1% of the phenotypic variation on these traits, respectively. The allele from Minghui 63 contributed to the increase of grain weight, grain length and grain thickness, but to the decrease of grain width. Moreover, the QTL also showed different modes of gene actions on the traits, such that partial dominance was observed for 1000-grain weight, grain length and grain thickness, while overdominance was detected for grain width.

TABLE 3
Effects of the QTL (in the interval GS09-
MRG5881) on grain shape and weight

| Traits | LOD | A″ | D^{b} | Var. %^{c} |
|---|---|---|---|---|
| 1,000-grain weight (g) | 72.8 | −4.08^{d} | −2.52^{d} | 83.4 |
| Grain length (mm) | 129.2 | −1.47^{d} | −1.06^{d} | 95.6 |
| Grain width (mm) | 8.9 | 0.05^{d} | 0.08^{d} | 19.8 |
| Grain thickness (mm) | 5.3 | −0.04^{d} | 0.004^{e} | 12.1 |

^{a} Additive effect of Chuan 7 allele
^{b}Dominance effect of Chuan 7 allele
^{c}Percentage of total phenotypic variance explained by the QTL
^{d}Significant at P < 0.0001 in t test
^{e}Not significant at P < 0.05

### 4. Progeny Test

Each plant in said random subpopulation was bred to 20 families (BC₃ F₃) which were subjected to progeny test. Progenies of 56 in 201 families were uniformly long grains, while 61 families were uniformly short grains and 84 families had both long and short grains. The ratio of the three groups fit well to the expected ratio (1:2:1) of single locus Mendelian segregation (χ2=5.67,P>0.05) in the χ2 test. The results indicated that in this BC₃ F₂ subpopulation, the grain size was controlled by a major gene, and the small size allele is dominant over the large size allele. The three distinct phenotypic classes corresponded to the three genotypes of the BC₃ F₂ individuals at the GS3 locus: homozygote for the Minghui 63 allele (long grain), homozygote for the Chuan 7 alleles (short grain), and heterozygote. Using the three phenotypic classes as a marker, GS3 was directly mapped into a 1-cM region delimitated by an Indel marker GS09 and SSR marker MRG5881 (FIG. 4).

### Example 3

### High Resolution Mapping of GS3

### 1. CAPS Analysis

9 CAPS markers used for the high resolution locating are listed in Table 2. The amplification product amplified by said markers had a size of around 1-kb. The PCR reaction system was identical with the SSR reaction system mentioned above. The amplification condition of the PCR was as below: 94° C. predenature 4 min; 94° C. 1 min, 53° C.˜57° C. 1 min, 72° C. 1.5 min, 34 cycles; 72° C. elongation 10 min. The digestion of the amplicons was carried out in a 20 μl reaction system containing: 10 μl PCT product, 1 U restriction digestive enzyme (from Takara Ltd., Japan). Additional components were as described in the manual provided by Takara Ltd. After digestion in 37° C. for 3-5 hours, 10 μl of the digestion product was subjected to separation by electrophoresis in a 1.5% agrose gel, which was then observed using UV after EB staining.

### 2. Analysis of the Recombinant Plant and High Resolution Mapping of GS3

To further narrow down the GS3 containing genomic region, 1,384 plants with long grain phenotype (long grain, 9.7 mm or longer) from the BC₃ F₂ population of 5,740 individuals derived from BC₃F₁-19 individual plant were selected for recombinant screening.

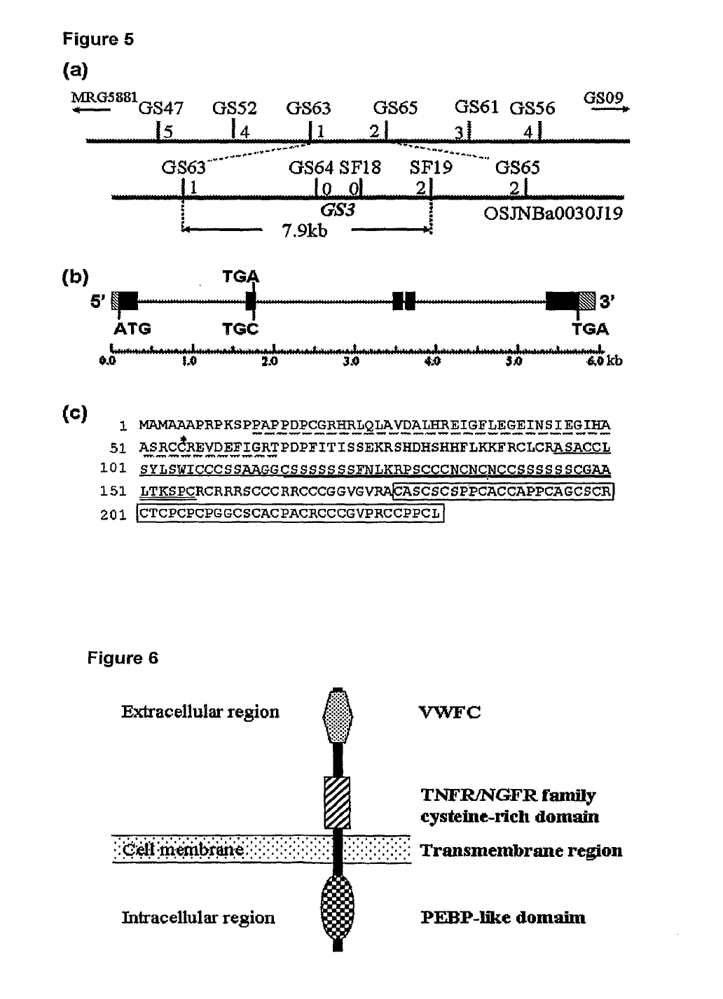

All of the 1,384 selected plants were screened using an SSR marker MRG5881 and Indel marker GS09, which identified a total of 55 recombinants which were further confirmed to be very large size singles by progeny test. Using 9 designed CAPS markers to screen the 55 recombinants, it was found that five recombination events were resolved between GS47 and GS3, four identified between GS52 and GS3, four between GS56 and GS3, three between GS61 and GS3, and two between GS65 and GS3 (FIG. 5a). In particular, the assay revealed one recombination event between GS63 and GS3 and two recombination events between SF19 and GS3. In addition, GS64 and SF18 were found to co-segregate with the GS3 locus. Therefore, the genomic region containing the GS3 locus was narrowed down to the DNA fragment bounded by GS63 and SF19, which corresponded to approximately 7.9-kb in length in the genome sequence of Nipponbare and 93-11 (FIG. 5a).

### Example 4

### Gene Structure and Predicted Function Analysis of GS3

### (1) Gene Structure Analysis of GS3

A full-length cDNA, which is named osigcea013f09t3, from the plumule of an indica cultivar Guangluai 4 (provided by Shanghai Agrobiological Gene Center) was identified in the 7.9-kb fragment (FIG. 4b) between GS63 and SF19 (which were designed by the applicants; refer to Table 2). The nucleotide sequence is shown in SEQ ID NO:1. The cDNA sequence of the cloned GS3 gene of the present invention is 953 bp in length, which matches well with the region between positions 1.6 and 7.3 kb of the 7.9-kb fragment. Allowing for the regulatory regions on both ends, this is considered as the only candidate gene for GS3.

The structure of GS3 gene was obtained by comparing the sequences of said total cDNA with the genomic DNA sequence of Nipponbare. GS3 gene is 5,363 bp in length from the translation start codon to the termination codon. It comprises 5 exons and 4 introns. The starting exon is 117 bp in length, while the second exon is 53 bp, third exon 45 bp, fourth exon 54 bp, terminal exon 430 bp in length, respectively. The first intron is 1,472 bp in length, while the second intron is 1,439 bp, third intron 83 bp, fourth intron 1,671 bp in length, respectively (Table 5b). Therefore, the open reading frame of GS3 gene is 699 bp in length and encodes 232 amino acids. The sequence of said gene is shown in SEQ ID NO:1.

### (2) Function Prediction of GS3

Prediction for the structure of GS3 protein was carried out with InterProScan. It was revealed that the protein encoded by GS3 gene consists of 232 amino acids and comprises several conserved domains. There is a PEBP (phosphatidylethanolamine-binding protein)-like domain in amino acid 12-65 at the 5′ terminus. A transmembrane region is located at amino acid 97-117. The region of amino acid 116-1557 is a TNFR (tumor necrosis factor receptor)/NGFR (nerve growth factor receptor) family cysteine-rich domain. The 3′ terminal cysteine-rich region shows the characters of the conserved amino acid sequence of the von Willebrand factor type C (VWFC) domain, which is typically 60-80 amino acid in length and comprises ten cysteines, especially is characterized in that it contains a C2XXC3XC4 sequence located in the middle and a C8C9XXC10 sequence at the 3′ terminal end (wherein C represents cysteine; the number represents the order of said conserved cysteines; X indicates any amino acid) (FIG. 5c and FIG. 6).

VWFC domain is represented in a number of extracellular matrix proteins. Some studies show that VWFC binds to members of the transforming growth factor TGF-β superfamily, thus disrupting the receptor binding sites of TGF-b superfamily proteins and preventing activation of the TGF-b receptor, such that it regulates the growth factor signaling pathway (Abreu et al., 2002, Gene 287:39-47; O'Leary et al., 2004, J. Biol. Chem. 279:53857-53866).

### Example 5

### Detection of Base Pair Variation Between GS3 Alleles by Sequence Alignment

### (1) Sequencing

Two large grain species (Minghui 63 and H94) and two small grain species (Chuan 7 and Zhenshan 97) were sequenced in the target genomic DNA region. DNA fragments from these cultivars were amplified using 10 pairs of primers whose amplicons were partially overlaping with each other. The amplification was carried out with high fidelity LA-Taq (TakaRa, Dalian, China). The PCR products were cloned into pGEM-T vector (Promega, USA) according to the manufacturer's specification. The cloned product was transformed into E. coli DH10B (Invitrogen, USA) and positive clones were screened with blue-white methods. The T7-R and SP6-F universal primers (Shanghai Sangon Biological Engineering Technology and Services Co. LTD.) and the Big Dye Terminator Cycle Sequencing v3.1 (Applied Biosystems, Foster City, Calif., USA) were used for sequencing from both ends of the subclones. Sequence contigs were assembled using the computer program SEQUENCHER 4.1 (Gene Codes Corporation, USA).

TABLE 4
Primers used in the sequence alignment.

| | | | Size of | Annealing |
|---|---|---|---|---|
| Marker | Forward primer (5′-3′) | Reverse primer (5′-3′) | Amplicon | Temp. (° C.) |
| GS63 | AAGAACGACTACGCGCATCT | CCATCGCTCTCTTTCCTCAG | 704 bp | 53 |
| | (SEQ ID NO. 16) | (SEQ ID NO. 17) | | |
| SF26 | GTCTGAGGAAAGAGAGCGAT | AAGCAAGCCAAGGGAAATGT | 1091 bp | 60 |
| | (SEQ ID NO. 26) | (SEQ ID NO. 27) | | |
| SF15 | AGCAAAAAAGGTGAAGGACG | CAAAGGGAATAACAAGGCAG | 1295 bp | 57 |
| | (SEQ ID NO. 28) | (SEQ ID NO. 29) | | |
| SF16 | CGAATAGGAAGTCAATGGC | GTCGTACCCGCCTTAGTTGA | 1159 bp | 55 |
| | (SEQ ID NO. 30) | (SEQ ID NO. 31) | | |
| SF28 | TGCCCATCTCCCTCGTTTAC | TGTTCGTTGCTGGTGTTG | 1065 bp | 55 |
| | (SEQ ID NO. 32) | (SEQ ID NO.33) | | |
| GS64 | CAACACCAGCAACGAACAAC | ACGAGGGATTATCAGCCATT | 1155 bp | 55 |
| | (SEQ ID NO. 18) | (SEQ ID NO. 19) | | |
| SF18 | CCTTCAGTAAGAGAGATGTG | AGTTGATGGTTTTGTGGGAT | 1245 bp | 57 |
| | (SEQ ID NO. 22) | (SEQ ID NO. 23) | | |
| SF45 | AACCTTCTCTTCCTACCCTT | TCAGCAATCACGTACTCATC | 1137 bp | 55 |
| | (SEQ ID NO. 34) | (SEQ ID NO. 35) | | |
| SF19 | TCTGCTTGCGGTTATCTGTA | TTAGGTCCCTTTTCTCGTCC | 1224 bp | 57 |
| | (SEQ ID NO. 24) | (SEQ ID NO. 25) | | |

The size of the PCR products depended on the sequence of Nipponbare and varied among different species.

### (2) Sequence Alignment

Sequence alignment was carried out on three large grain varieties (Minghui 63, H94, 93-11) and three small grain varieties (Zhenshan97, Chun 7, Niponbare) (FIG. 2). The GS3 region sequences of Nipponbare and 93-11 were from the BAC clone OSJNBa0030J19 and contig Ctg009226, respectively. Sequence alignment was conducted using the computer program Vector NTI 9 (InforMax™ Corporation, USA).

Although many nucleotide changes were observed among the six cultivars in the 7.9-kb region, there was only one common single nucleotide mutation detected between these two different grain-length groups, which indicated said mutation was the essential reason causing the grain size change. Further studies showed that said nucleotide mutation was located at the second exon of the GS3 gene, in which a cysteine codon (TGC) in the small-grain group was mutated to a termination codon (TGA) in the large-grain group (FIG. 4b). This premature termination resulted in a 178-amino acids truncation in the C-terminus of the predicted protein in the large-grain group, which eliminated part of the PEBP-like domain and all the other three conserved domains. Such nonsense mutation was clearly in agreement with the recessive nature of the long grain phenotype, indicating that long grains resulted from the loss of the function of the protein otherwise producing short grains.

What is claimed is:

1. An isolated polynucleotide comprising a nucleic acid sequence selected from the group consisting of SEQ ID NO: 1 and SEQ ID NO: 2 or complements thereof.

2. A recombinant DNA construct comprising a polynucleotide according to claim 1.

3. A recombinant DNA construct according to claim 2, wherein the polynucleotide is operably linked to a promoter functional in a plant cell.

4. A recombinant DNA construct according to claim 2, wherein the polynucleotide is operably linked to a 3′ untranslated region functional in a plant cell.

5. A transformed cell or organism comprising a polynucleotide according to claim 1.

6. The transformed cell or organism according to claim 5, wherein the cell is a plant cell or plant.

7. The transformed cell or organism according to claim 6, wherein the organism is a plant selected from the group consisting of cotton, wheat, soybean, maize, rice, and canola.

8. A substantially purified polypeptide comprising the amino acid sequence of SEQ ID NO: 3.

9. An isolated polynucleotide encoding a polypeptide having an amino acid sequence of SEQ ID NO: 3.

10. An isolated polynucleotide encoding a polypeptide having at least 70% amino acid sequence identity with a polypeptide having an amino acid sequence of SEQ ID NO: 3.

11. A recombinant DNA construct comprising a polynucleotide selected from the group consisting of:
(a) a polynucleotide comprising a nucleic acid sequence selected from the group consisting of SEQ ID NO: 1 and SEQ ID NO: 2;
(b) a polynucleotide encoding a polypeptide having an amino acid sequence of SEQ ID NO: 3;
(c) a polynucleotide comprising a nucleic acid sequence complementary to a nucleic acid sequence selected from the group consisting of SEQ ID NO: 1 and SEQ ID NO: 2;
(d) a polynucleotide having at least 70% sequence identity to a polynucleotide of (a), (b) or (c);
(e) a polynucleotide encoding a polypeptide having at least 70% sequence identity to a polypeptide having an amino acid sequence of SEQ ID NO: 3;
(f) an oligonucleotide comprising from about 15 to 100 nucleotide bases, wherein the oligonucleotide hybridizes under high stringency conditions to a polynucleotide of (a), (b) or (c);
(g) a polynucleotide comprising a promoter functional in a plant cell, operably joined to a coding sequence for a polypeptide having at least 70% sequence identity to a polypeptide having an amino acid sequence of SEQ ID NO: 3, wherein the encoded polypeptide is a functional homolog of the polypeptide having an amino acid sequence selected of SEQ ID NO: 3; and
(h) a polynucleotide comprising a promoter functional in a plant cell, operably joined to a coding sequence for a polypeptide having an amino acid sequence of SEQ ID NO: 3, wherein transcription of the coding sequence produces an RNA molecule having sufficient complementarity to a polynucleotide encoding the polypeptide to result in decreased expression of the polypeptide when the construct is expressed in a plant cell.

12. A transformed plant comprising a recombinant DNA construct, wherein the construct comprises a promoter region functional in a plant cell operably joined to a polynucleotide comprising a coding sequence for a polypeptide having an amino acid sequence of SEQ ID NO: 3.

13. A transformed plant of claim 12 wherein the polynucleotide is oriented with respect to the promoter such that transcription of the polynucleotide produces an mRNA encoding the polypeptide.

14. A transformed plant of claim 12 wherein the polynucleotide is oriented with respect to the promoter such that transcription from the polynucleotide produces an RNA complementary to the mRNA encoding the polypeptide.

15. The transformed plant according to claim 12, wherein the plant is selected from the group consisting of cotton, wheat, soybean, maize, rice, and canola.


SEQUENCE LISTING (35 sequences)

SEQ ID NO: 1 - DNA, 7883 bp, Oryza sativa
  gene (1)..(7883)
  5′UTR (1609)..(1645)
  exon (1646)..(1762)
  exon (3235)..(3287)
  exon (4726)..(4770)
  exon (4854)..(4907)
  exon (6579)..(7008)
  3′UTR (7009)..(7222)
gccgccatgagctcaccggtgctgccgccgacctcctgcctacgtgccgccttctccgctgcaagtcgccgctgctgccctccccacgacacagctggatccgggaggggagggcagggaaggttcgctgtcgccgccgccaccctccctacgacgtagccagatatgggtggagaggtaagcgccgccgccgctgcacaacccacgatgccgccgctgcacaacccgcgatgccgtcggccggatttgggaggtggagcgtcgccgccctccgccccgcgccatgtctgaggaaagagagcgatgggagcgccgccggccaccaccgtccccccttccccctccaaccagatctgggaggaagggagggagggaggggagccatcacgtcgttgggagtagatgccgccaccgccctccccctccctgcggccaccacactggggtgcaccgccaccgctaggtagccgcctctggctggatccacgccgaggagaggaaggagagggagagggagagggaggggaggggagatgtggaagttgacttagaaaattttgacgcccggtggtttttaagatactttacctttttgcaggcggatttattaaagaggtccgtctgcaaaaataatgatattttcacggtcggacctcttaagatatctacatgtagaaatctatatttttttacttaaattactgccagggctttgcctcgtagtataattttcataaaaactcaaaatatttacaaattacaaaagaggaaggaaggagaaaaacttataaagattacaatgtttgtaaccaatcaaaaactatctgctttaaaggatctttcattctacacatatgaagaagaacttcttttctaaacgatattcttcatgaaccaaaggagggaagctcattccgaaaatttttcccatttctctgctcgtaggcggataactatctcatctcacggttgttatttttgtaggtggctatttggcttccagagacctactcgtccgggaaaaagaaatgccagcatctagaaaaatagtttttctagtagagtgttgtgagcgtctgcgatcgtggtaaagaaagcaaaaaaggtgaaggacgagtggcatgatacatatgggaaagctgtagactttgacccttgactactcgttggaagtgtgcgtctgcatgcattattgaacggctctgatccccgcggcgcagcggatcggggtcatgtccggatgggcatatcgacgagaaggatccgtccccgacaatctttcaaggcccgtgcccccgtccctcctctcctctgcgcctttccatcatcatttacgcccaaccccaacacatgtacatttcccttggcttgcttccggagaagaaaagagcggccatccactccactctccactctctcccttccatcattacttgcccaaaaacggcaatcccctcccctccatctccatgtgctcttccacctagctccgccattcaaagcaaagcaccaagcttttgcctctccctctaccatgcctgccccctatacatagctgctgcaccgtctctcttcataaatatactagtaggagtagcagagctcatcacttcgatcatctccattatcggaacttcggagtgacatggcaatggcggcggcgccccggcccaagtcgccgccggcgccgcccgacccatgcggccgccaccgcctccagctcgccgtcgacgcgctccaccgcgagatcggattcctcgaggtacaatctatctctatctgtctatatcactaccattcatactccttcgatcttgcttcaaaacaaaaaaatatatatttcctacttcatattcatatacacacgtacggcttgctatctgtcgaattgtttgcttctgcatgcatgcatcactctcattgtaagtttttcccagcttaaaaccactccttttatcttcgttcttcttccttcttgtttttttttaaaaaaacaacaactcatttaatcttcatatagtgtatcatgcatcatttgcttctttgatcagttccccaaaaactgctcctctcttcccagccaaataattaaacttaagcaaacaagcaagttgaactgatgatccaataaaacaaaacaaaaccgatcgaataggaagtcaatggcatataccagctgctatagctgaagccacgaatgcttagcttagctctagtcgatccctgttgactgttcaacacactgcactaacacaccagttaatgagctgattaattaaaccattaaatgagcttaacgggcggctagcttcttcctctgggcccgtgccgatcgtaccatcggtttgcgcgtccctccacctaaactctctgccttgttattccctttgcctagtactacatgcatttgcatcatcatcccatcaccaatagtactacgtttcaactggattttggtggtgtccaaccatatcatatttggttttgttctctagtttactcctacattagtctctagcggttttgtggagtactaaagaaaacaactaatcagccagggtttaacgtttaatcggttggtggttttgttaattaatttcatctactattttagacttcacaggtcttcgagctataagcatcgattgccatgcatcaatcgatgctggtccacgctagtttctgagttctgactagctctcttaattgtgctttgacctactttaattaattaaccagtggctgcgtcactcattgaccaacattgtcatgttacccggactgattttttttttctttaaaaaaacaccggatatattattagttagtgtatatatatgtctgctcaagaagcgcatgcatatagtttctcgtcaaacaaaaaatgtactgtatgctcaaagcatctgttttggaattgtcatattcgcctttataattaaaataattaaaatggtgatgcccagctttttttttcctccaataatttatttattggcttgatttcctgtgctattaggagtaaaactactccgttttaattagcaccatttttaaagcttctaaaattaacctaagtaaagtacgacagtacttgctgtctagctttaaatgttttgggtgttaaaatatccctcagacatcacctgaaaagttgacaggctaaacacatgcccatctccctcgtttacttaaattaattcgaacaaacaactgtatatatatttcttgcagggtgaaataaattcaatcgaagggatccacgctgcctccagatgctgcagagagtaagccagcctgctgtttctttttgtactacttccatttcttctcgtctttactcttaccatgcattcacaaaatatacttacttaccccagtttttgatcatgaactttgaccgttatctttttaagcaacttcaataaaataggtttaaatgcaaatgttatgtacaccattgattataaaaccttggcaaatgaaagtaaaaaaccagcacatttaatttctgaacgttgggagtattattatttttatatcttttactatcatttaatcatagtatcgtgcaagctttttgagtgtaattaggttgcttaaggtaaaaaaatgtaactaggttacatttagtactaaactgaacatttaattagtaatgtttcgttaagtaactgtaatttcaatgcatgcatgtcctcccgtaagagcaagtttaatagtatagccaactactagctccaatttatttatagacaatctaatagctcattcatacaataattacatactacactattaatatctgatcccacctgtcatacacatactgcattttggagtccgtgctatagctgactacaaatctatagtccgctgctcttctctctctttatttatctccttaaaatatgtttgcagctggcttatagcctgctattgtacctgctctgaaaatagtgcagagactgttcaaaaagtcattgcacaataactattcacatggaactgtgaaaagtatatattggaacttactagctagatccttttgggaacatgggaaaagccaagtcacgtgtggaatccctattccctgtgttcttcagctagaagagtgaaaataatgtactactactatacggagatgaaattacagcaggagcagaaagcgggaaaaaaactttaaatcaattaaacaaacctctctctgcaaaattcaacaccagcaacgaacaactcatcaagttcttgtgttatgtaccggccggtaactaattgttgtttgcataagcgaaacggtatatttgcaaacaaaaaataatttatgaataaaacttttatatacatgttcttaattatctcaaaacaaaggttgaaaaataaacttcgatgaaaaaatctcaaaatcaattccaaatttatggtgaaaattttaaattttgtctgataaacataagtataagcaaaaaaaaaagtaaagcaatgtcactatgttaatggtattggtatatatacctttgagtttctgtctgtactttaggagtacatgctacgaacatgttttcttggcttctatttcgttggaattacttgcgtattgtgggccagacgcctggtgaacttcgtcgattgtgtggactaattaagctcacctgaaataagtggttgaaaacaaggctaaagatgattttaatcatggattttggcttggaaatttttttgtagggttgacgaattcatcggaagaactcctgatccattcataacgatgtatggattttcaggtcgagaatttgtctttaacttcgcacgactgttattttttttcttattaattctctgtttacaagcagttcatcggagaagcgaagtcatgatcattctcaccacttcttgaagaagtttcggtactcacttcattcccggatcttaatgtatatatgcatatctgcactgtgctaattggtgtacacattatgtgatcatcagtccaagttaattattacttacaaaactgaactaataaacactagaaaatatgtaacttgcaaagtacatattgaatcagggattcatatatagaactccacctgcagatttcttccaatatatatatgctgtcaccatgttttcacttgtcacctagtacacctttgactgggagactttccttgatgatcgacgtggtcatattcttcagattgatttaatttcagatagaaaaaaatattgtttacttagtttctctccttcagtaagagagatgtgcaagaccagcgatcaaactatatgaactgttcgtttcatgataaaaaaaacatgatatggaataactaggtgattcaacatataatggctgataatccctcgtttcaggagataccatcagttttctacttttctacttttctccatgttctctttttcatgtttgaggtggatcggagcttgtattagatgtttgctcagctcaattattgctgcagatttccctatatagcctccactgtatatatactccctccgattccataatttaatgatattttgaacaatgacgctgtctccaaaatatatctttcactttgttttcctattataatatatacaataaaaaaaatacatatttacttttcttataaatagtttcaaagacaaatctatatatgttgttatataactcttttaaactaaatatttttaaagttatagtcaaagttacaaaagttggacctcaaacatgtataaaacgtcgagaattgtatatctgatcaaccaataatttgtagtgcattgtcttaaaaaaattgcatttctagctatgtatctagataattacaagaaccaggtgaaatcacattttatttttactggaccacgaactcattgtttaattacttccagccttgcactaaataacaataattgaacctggcatcacctgcacaattaatttggacacaaagtaaacatgaatgcaacatacttcgtttcatattgtttgttggtgtaggatttaaattttgtgtcaaaatacttgccgttcttactacattcttaagcactttgagaactaaccttctcttcctacccttcatcaatacagtcatactaactaattggctcttatgccttgaaaaactaattaggatgtatttaatgagggtaaccatgtaatctgccaccagtgaatgcaattttggttcaaaatttcgggcccccgccctaaaaagtcattatctcgatagaatttttttgaatttagtcaaaatttattcaaatttagccaaattgtgttaaatttcaaataatttcagtctaaaaagtgctgaaaatcccgaaatttaggttctaccgaaatggccggaaattttcagcgaaaatcaaacatgaaaaccttgtctgccacatttgttagtttgtgtaaagatttgctagaacgataagtaatttgaagcggatgtatattatatatcccacaaaaccatcaacttgttaattacatgtatatttgtgcatgatgctttcaccactttgtggttgttaacgattaaatcacacgttttattttctcacagctgtttgtgcagagcaagtgcgtgctgcctcagctacctctcctggatctgctgctgcagcagcgccgccggcggctgctcatcctcctcctcctcctccttcaacctcaagaggccgagctgctgctgcaactgcaactgcaactgctgctcctcctcctcctcctcatgtggggcggcgttaacgaagagtccgtgtcgctgccgccgccgcagctgctgctgccgtcgctgctgctgcggcggcgtcggcgtccgcgcgtgcgcgagctgcagctgctccccgccgtgcgcgtgctgcgcgccgccgtgcgcgggatgctcgtgccgctgcacctgcccgtgcccgtgccccggcggctgctcctgcgcgtgcccggcgtgcaggtgctgctgcggcgtccctcgttgctgccccccctgcttgtgatcgatcgatcgattgagcgaagctgcactgattggttaattaattagttctcgatgatgatcgatcgagctgcgcgcgtacttaattagctagctaggttctggtgttaattagttcctcatcgatgcatatgttgattgccttgctctgcttgcggttatctgtaatttggctttgctgccatgatgagtacgtgattgctgatttattttacatatcctctgctatatatatctagctggtagtagctagttttgatctcacttgcaaaactattcatttcctgattttaacaaggtcaaatgacttggttgcctttggttcatactccttagagcaagtataaaggtttatatggaggagagaggtaacaaaaaaaaatcaagagtattggctctcatgcgagagctagcttatcacaagctacaaatcaaatatattaaatgtataagtaagagatagagagaggaataaaattatagctaaccttataggtaatgtattatatatgttaattttaaaataagctaatagtaaaaagtgagctttattattatccttgctcttagtaatggcctcgattcctacattttttttttgagagagtagtacatattttcgcagcgtaatatggacattggccgacgggcctaactattactgggtctaagtgcctaactgagctaaaagccttaaacccaatactgaaaacgaaaaagttcactccgggtccctcatcttgtcgacgggttttaaaatcatcattgaaccggaatacgcattcccaatctttcaaaaccggatcacactaggtctaaggcagtattgctcccgattttggctgatatggtggttgagtcagcgtgagacacaca

SEQ ID NO: 2 - DNA, 699 bp, Oryza sativa
  exon (1)..(699)
atggcaatggcggcggcgccccggcccaagtcgccgccggcgccgcccgacccatgcggccgccaccgcctccagctcgccgtcgacgcgctccaccgcgagatcggattcctcgagggtgaaataaattcaatcgaagggatccacgctgcctccagatgctgcagagaggttgacgaattcatcggaagaactcctgatccattcataacgatttcatcggagaagcgaagtcatgatcattctcaccacttcttgaagaagtttcgctgtttgtgcagagcaagtgcgtgctgcctcagctacctctcctggatctgctgctgcagcagcgccgccggcggctgctcatcctcctcctcctcctccttcaacctcaagaggccgagctgctgctgcaactgcaactgcaactgctgctcctcctcctcctcctcatgtggggcggcgttaacgaagagtccgtgtcgctgccgccgccgcagctgctgctgccgtcgctgctgctgcggcggcgtcggcgtccgcgcgtgcgcgagctgcagctgctccccgccgtgcgcgtgctgcgcgccgccgtgcgcgggatgctcgtgccgctgcacctgcccgtgcccgtgccccggcggctgctcctgcgcgtgcccggcgtgcaggtgctgctgcggcgtccctcgttgctgccccccctgcttgtga

SEQ ID NO: 3 - protein, 232 aa, Oryza sativa
MAMAAAPRPKSPPAPPDPCGRHRLQLAVDALHREIGFLEGEINSIEGIHAASRCCREVDEFIGRTPDPFITISSEKRSHDHSHHFLKKFRCLCRASACCLSYLSWICCCSSAAGGCSSSSSSSFNLKRPSCCCNCNCNCCSSSSSSCGAALTKSPCRCRRRSCCCRRCCCGGVGVRACASCSCSPPCACCAPPCAGCSCRCTCPCPCPGGCSCACPACRCCCGVPRCCPPCL

SEQ ID NO: 4 - DNA, 20 bp, Artificial Sequence
  Synthetic Primer
agcaaagctggaacgaagag

SEQ ID NO: 5 - DNA, 20 bp, Artificial Sequence
  Synthetic Primer
taaattacgccgtgtcaacg

SEQ ID NO: 6 - DNA, 20 bp, Artificial Sequence
  Synthetic Primer
gcaaccaagtccacgctaat

SEQ ID NO: 7 - DNA, 20 bp, Artificial Sequence
  Synthetic Primer
tagccgaagatcagcctcct

SEQ ID NO: 8 - DNA, 20 bp, Artificial Sequence
  Synthetic Primer
gattattggagacgggacga

SEQ ID NO: 9 - DNA, 20 bp, Artificial Sequence
  Synthetic Primer
gacggcatgaccactctttt

SEQ ID NO: 10 - DNA, 20 bp, Artificial Sequence
  Synthetic Primer
agctttggtgtcgttctgct

SEQ ID NO: 11 - DNA, 20 bp, Artificial Sequence
  Synthetic primer
ccgacttggagagaatggaa

SEQ ID NO: 12 - DNA, 20 bp, Artificial Sequence
  Synthetic primer
gctgtgttgtcctttgctga

SEQ ID NO: 13 - DNA, 20 bp, Artificial Sequence
  Synthetic primer
ccaataaaccccactgcaac

SEQ ID NO: 14 - DNA, 20 bp, Artificial Sequence
  Synthetic primer
ctttacaaaaccggcggtaa

SEQ ID NO: 15 - DNA, 20 bp, Artificial Sequence
  Synthetic primer
tgaagcggacctagcatttt

SEQ ID NO: 16 - DNA, 20 bp, Artificial Sequence
  Synthetic primer
aagaacgactacgcgcatct

SEQ ID NO: 17 - DNA, 20 bp, Artificial Sequence
  Synthetic primer
ccatcgctctctttcctcag

SEQ ID NO: 18 - DNA, 20 bp, Artificial Sequence
  Synthetic primer
caacaccagcaacgaacaac

SEQ ID NO: 19 - DNA, 20 bp, Artificial Sequence
  Synthetic primer
acgagggattatcagccatt

SEQ ID NO: 20 - DNA, 20 bp, Artificial Sequence
  Synthetic primer
cggtatgccaagttgaatga

SEQ ID NO: 21 - DNA, 20 bp, Artificial Sequence
  Synthetic primer
ttgccgcagtaaacaagaag

SEQ ID NO: 22 - DNA, 20 bp, Artificial Sequence
  Synthetic primer
ccttcagtaagagagatgtg

SEQ ID NO: 23 - DNA, 20 bp, Artificial Sequence
  Synthetic primer
agttgatggttttgtgggat

SEQ ID NO: 24 - DNA, 20 bp, Artificial Sequence
  Synthetic primer
tctgcttgcggttatctgta

SEQ ID NO: 25 - DNA, 20 bp, Artificial Sequence
  Synthetic primer
ttaggtcccttttctcgtcc

SEQ ID NO: 26 - DNA, 20 bp, Artificial Sequence
  Synthetic primer
gtctgaggaaagagagcgat

SEQ ID NO: 27 - DNA, 20 bp, Artificial Sequence
  Synthetic primer
aagcaagccaagggaaatgt

SEQ ID NO: 28 - DNA, 20 bp, Artificial Sequence
  Synthetic primer
agcaaaaaaggtgaaggacg

SEQ ID NO: 29 - DNA, 20 bp, Artificial Sequence
  Synthetic primer
caaagggaataacaaggcag

SEQ ID NO: 30 - DNA, 19 bp, Artificial Sequence
  Synthetic primer
cgaataggaagtcaatggc

SEQ ID NO: 31 - DNA, 20 bp, Artificial Sequence
  Synthetic primer
gtcgtacccgccttagttga

SEQ ID NO: 32 - DNA, 20 bp, Artificial Sequence
  Synthetic primer
tgcccatctccctcgtttac

SEQ ID NO: 33 - DNA, 18 bp, Artificial Sequence
  Synthetic primer
tgttcgttgctggtgttg

SEQ ID NO: 34 - DNA, 20 bp, Artificial Sequence
  Synthetic primer
aaccttctcttcctaccctt

SEQ ID NO: 35 - DNA, 20 bp, Artificial Sequence
  Synthetic primer
tcagcaatcacgtactcatc